(19) United States
(12) Patent Application Publication
(10) Pub. No.: US 2008/0039405 A1
(43) Pub. Date: Feb. 14, 2008

(54) Modification of percutaneous absorption of topically active materials

(75) Inventors: Nigel A. Langley, Belle Mead, NJ (US); Abel G. Pereira, Bridgewater, NJ (US); Laurie B. Joseph, Monroe Township, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Croda, Inc., Edison, NJ (US)

(21) Appl. No.: 11/788,759

(22) Filed: Apr. 20, 2007

Related U.S. Application Data
(60) Provisional application No. 60/794,602, filed on Apr. 25, 2006.

(51) Int. Cl. A61K 31/075; A61K 31/01; A61K 31/185; A61K 31/35; A61K 31/415; A61K 31/70; A61K 31/56; A61K 31/355; A61K 31/315; A61K 31/07

(52) U.S. Cl. 514/29; 514/169; 514/396; 514/456; 514/458; 514/494; 514/576; 514/714; 514/717; 514/725; 514/762

(57) ABSTRACT

The present invention relates to methods of influencing the flux or surface retention time of a topically active pharmaceutical ingredient through skin and formulations relating thereto.

### CROSS-REFERENCE TO RELATED APPLICATIONS

This application claims the benefit of the filing date of U.S. Provisional Application Ser. No. 60/794,602 filed Apr. 25, 2006, which is hereby incorporated by reference.

### BACKGROUND OF THE INVENTION

There are numerous topical pharmaceutical products, which are applied to the skin to treat various conditions. Unfortunately, many of the pharmaceutically active ingredients provided in topical formats can actually penetrate the skin too quickly. This can have a number of potentially adverse consequences. First, the actual degree of exposure of active ingredient and the fungus, bacterial infection or other skin condition in any given skin layer may be too brief. This can require additional dosing, higher dosing frequencies and prolonged treatment. In extreme cases, a particular product could be rendered ineffective.

Second, when the active ingredient traverses the skin, it may enter the bloodstream where it may be active on unintended vessels and organs. The faster the active material traverses the skin, the greater the amount which reaches the bloodstream unaltered—not having interacted with the intended condition—and hence is available to other systems and organs within the body. Therefore, it would be desirable to provide formulations, which prolong the length of interaction between an active ingredient and the actual layers of skin. Certain fatty alcohol phosphate ester mixtures and their use in topical products are disclosed in U.S. Pat. No. 6,117,915, issued Sep. 12, 2000, to Pereira et al. See also K. F. Gallagher, A New Phosphate Emulsifier for Sunscreens, 113 Cosmetics & Toiletries, 73 (1998).

### SUMMARY OF THE INVENTION

The present invention can provide advantage hitherto unrealized in the field of topical pharmaceutical preparations. By use of the present invention, one can retard the flux (the rate at which a specified amount of a material applied to a specified surface area of skin traverses or travels across the skin in a given period of time) of a topical active pharmaceutically ingredient or “TAPI.” By retarding their transport, i.e., by decreasing their flux across (through the skin—from one side to the other), the active ingredients are provided with more opportunity to interact with the patient's skin condition in the afflicted area. This may permit more efficient, therapeutic relief. In certain embodiments, this may lessen: the length of treatment; the amount of active which must be applied in any one application or in total; the frequency of application; and/or the amount of active which traverses the skin entirely and becomes bioavailable through the circulatory system. Thus, in one embodiment, the present invention provides methods of decreasing the flux of a TAPI across the skin by formulating the TAPI in a topical formulation including mixed fatty alcohol phosphate esters. Methods of increasing skin retention time are also contemplated and include the same steps.

In another embodiment, the present invention involves a method of improving the “skin retention time” or decreasing the flux of a TAPI in an already known product comprising the steps of adding to that product an effective amount of a fatty alcohol phosphate ester or mixture in accordance with the present invention and forming a homogenous mixture therewith. The improved formulations resulting from that addition are also contemplated.

Methods of treating humans and animals in need of such treatment by applying to at least one afflicted area of the skin a composition in accordance with the invention that exhibits a decreased flux or increased skin retention time, particularly compared to an otherwise identical formulation not containing mixed fatty alcohol esters of the invention for a time sufficient to provide a biological effect are specifically contemplated. A “biological effect” does not mean that condition is effectively treated, cured or prevented—or even that symptomatic relief is obtained. Moreover, a biological effect may not be observed or measurable for days or even weeks, even with repeated applications of products in accordance with the present invention. A biological effect can be observed on the cellular level, at the level of a layer of skin or in gross and includes any change in biological system that is eventually observable or measurable that results directly or indirectly from the application of the TAPI.

In another embodiment there are provided new topical formulations containing at least one TAPI and a mixed fatty alcohol phosphate ester in accordance with the present invention. In one preferred embodiment in accordance with this aspect of the invention, these formulations exist as an oil-in-water, water-in-oil or oil-in-oil systems.

In another embodiment, there are provided formulations containing an amount of mixed fatty alcohol phosphate esters that are sufficient to reduce the flux or increase the surface retention time of the TAPI through the skin, when measured after a period of 24 hours and when compared to the same formulation which omits the fatty alcohol phosphate esters. In a preferred embodiment, these topical formulations reduce the amount of TAPI which traverses the skin after a 24-hour period by 10% or more as measured by counts per minute or CPM or on a weight basis as assayed when compared to the identical formulation without mixed fatty alcohol phosphate esters as described herein. Methods of making these topically active formulations and methods of their use are also contemplated.

Finally, and in an alternative embodiment, there is provided a method of administering a drug to the blood stream at a predetermined rate comprising the steps of applying a specific amount of a nontopically active pharmaceutical ingredient (“API”) to a predetermined surface area of a patient's skin, preferably a portion of the skin not afflicted with a condition for which the API is being administered to treat, for a time sufficient to allow the desired amount of the nontopically active pharmaceutical ingredient to traverse the skin and enter the blood stream. Formulations including the mixed fatty alcohol phosphate esters or mixtures of the invention along with a nontopically active pharmaceutical ingredient are also contemplated. The products and methods of the invention are suitable for both human and veterinary use and both are contemplated unless otherwise specified.

In one embodiment, the invention provides a topical pharmaceutical preparation exhibiting decreased flux or increased skin retention comprising: a topical active pharmaceutically ingredient in an amount of at least about 0.1% by weight of the final preparation, about 0.1 to about 20% by weight of mixed fatty alcohol phosphate esters comprising at least one alkoxylated fatty alcohol phosphate ester and at least one non-alkoxylated fatty alcohol phosphate ester present in a ration of 80:20 to 20:80 and a vehicle, said preparation having an improved flux or increased skin retention time of at least about 10%, more preferably about 20% or more, in 24 hours (when measured at about 24 hours) when compared to the same preparation without said mixed fatty alcohol phosphate esters.

In another embodiment, the invention provides a pharmaceutical preparation exhibiting decreased flux or increased skin retention comprising: a active pharmaceutically ingredient in an amount of at least about 0.1% by weight of the final preparation, about 0.1 to about 20% by weight of mixed fatty alcohol phosphate esters comprising at least one alkoxylated fatty alcohol phosphate ester and at least one non-alkoxylated fatty alcohol phosphate ester present in a ration of 80:20 to 20:80 and a vehicle, said preparation having an improved flux or increased skin retention time of at least about 10%, more preferably about 20% or more, in 24 hours when compared to the same preparation without said mixed fatty alcohol phosphate esters.

The present invention also provides a method of treating a topical condition in a patient in need thereof by applying to an afflicted area of a patient the composition of claim 1, maintaining said composition in contact with said afflicted area of said patient, and optionally reapplying said formulation, for a time sufficient to treat said topical condition.

### BRIEF DESCRIPTION OF THE DRAWINGS

FIG. 1 illustrates the reduction in flux of 68% of 3H-cortisol in test skin between two identical formulations with (“CES”) and without (“no CES” or “S-70”) the mixed phosphate esters of the invention at 24 hours.

FIG. 2 illustrates the reduction in flux as illustrated in FIG. 1, at 48 hours-57% more 3H-cortisol in/on the test skin.

FIG. 3 illustrates the systems of FIGS. 1 and 2 at 72 hours—system has rendered equilibrium.

FIG. 4 illustrates the relative amount of 3H-cortisol in the receptor fluid after 24 hours of two formulations one with and one without a mixed phosphate ester of the invention-91% decrease in CES material in receptor fluid compared to S-70.

FIG. 5 illustrates the relative reduction of 3H-cortisol in the receptor fluid of the formulations illustrated in FIG. 4 after 48 hours—reduction of 68%.

FIG. 6 illustrates the relative reduction as in FIGS. 4 and 5 at 72 hours—reduction by 19%.

### DETAILED DESCRIPTION

While the specification concludes with the claims particularly pointing and distinctly claiming the invention, it is believed that the present invention will be better understood from the following description. All percentages and ratios used herein are by weight of the total composition and all measurements made are at 25° C. and normal pressure unless otherwise designated. However, PBS was kept at 37° C., by circulating water bath, so tissue was exposed basally to 37 and apically to RT. The present invention can “comprise” (open ended) or “consist essentially of” the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention. Preferably, such additives will not be present at all or only in trace amounts. However, it may be possible to include up to about 10% by weight of materials that could materially alter the basic and novel characteristics of the invention as long as the utility of the compounds (as opposed to the degree of utility) is maintained. All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

In accordance with the present invention, one objective of certain embodiments is increasing the “skin retention time” of a TAPI. By improving the skin retention time, it is understood that the term encompasses both the amount of time the TAPI is maintained on the outer surface of the skin and/or within any layer thereof. This is evaluated by determining either the amount of TAPI that remains on and/or within skin after 24 hours of exposure using the in vitro testing methodology described herein or by determining the amount of TAPI that has completely traversed the skin and is contained in a receptor fluid using the methodologies described herein including normal analytical assays. Increases in skin retention time, particularly on the outer surface of the skin, can also be observed by skilled medical professionals by simply observing the application area.

As long as the skin retention time of a topical formulation is improved (e.g., lengthened) when compared to an identical formula not containing mixed fatty alcohol phosphate esters, as described herein, in something more than a negligible way, that is considered improvement. However, in a preferred embodiment in accordance with the present invention, the degree of improvement is at least about 10%, more preferably at least about 20%, more preferably at least about 30% either by CPM or w/w depending upon the system used to measure. These are all based on measurements taken at 24 hours after the test begins.

Another term used herein, sometimes interchangeably with skin retention time is “flux.” Flux is a measure of the amount of API that traverses or moves across a predetermined area of skin when measured after a period of 24 hours in accordance with the methods described herein when compared to the same formulation, which omits the fatty alcohol phosphate esters or mixtures thereof as described herein. Again, this can be measured directly by measuring the amount of material in the retention fluid after 24 hours and subtracting it from the original amount applied to the skin. In addition, flux can be determined by calculation based on subtraction of the amount of TAPI left in the skin or on the skin subtracted from the amount initially applied and using standard analytical methods. As long as the flux (rate of transport) is lowered for a product in accordance with the present invention when compared to the flux for the product without the mixed fatty alcohol phosphate esters of the present invention, flux has been “improved” in accordance with the invention. Preferably, the amount of improvement is at least about 10% and more preferably about 20%, more preferably at least about 30% either by CPM or w/w depending upon the system used to measure. These are all based on measurements taken at 24 hours after the test begins. That means that the amount of active in the receptor fluid, after 24 hours, is 10, 20, or even 30% less because of the invention.

Note that in the context of both skin retention time and flux, any technique that provides reliable data can be used as a measure. It is not always practicable to measure the ability of a particular formulation to establish an improvement in either skin retention time or flux by reference to an actual API or TAPI found in a premade formulation. While sometimes the API or TAPI retained in or on the skin can be determined by an assay of the receptor fluid can be measured analytically at 24 hours. A marker or surrogate compound may also be added to a formulation in accordance with the present invention or any formulation that is being evaluated. In that case, the flux or skin retention time of that surrogate compound may be measured instead of or in addition to the actual API or TAPI. As described herein, one such surrogate is a radioactive cortisol known as ³H cortisol. This may be added to a given formulation and if the formulation tested with and without the mixed fatty alcohol phosphate esters of the present invention are compared, one can evaluate whether or not skin retention time or flux are influenced and indeed improved by the addition of the mixed fatty alcohol phosphate esters. The use of such markers or surrogates is a preferred way to determine flux or skin retention time in accordance herewith.

Note further that in certain embodiments of the present invention such as those concerned with the actual delivery of an API to the blood stream, skin retention time is not as important as flux. Delaying and/or controlling the delivery of certain active ingredients can be highly desirable. Thus, by reducing the rate of delivery (e.g., flux, the amount of API traversing a defined portion of the skin in 24 hours) one may be able to extend the release of a drug over a period of time that is considered desirable.

Both topical and systemic embodiments of the present invention can be used alone or in combination with other types of treatment. For example, a topically applied API that provides, in a given formulation in accordance with the invention, a desired flux, may be useful in combination with orally ingested dosage forms to assist in maintaining at least minimum blood level exposures.

While the invention may be described in terms of patients or subjects, a term which includes people, it will be appreciated that formulations in accordance with the present invention are also highly useful for the topical and systemic administration of APIs and TAPIs for veterinary use, principally in connection with mammals such as dogs, cats, rodents, horses, domestic livestock and the like. Indeed, in one embodiment, the present invention is formulated such that it can be applied to one area of the skin of an animal in treating a veterinary condition and the material will then spread over a much greater portion of the surface area of the animal skin.

Note also that in this instance, and indeed in other instances as well, the TAPI can be an insecticide such as the material used to retard or fleas and ticks or other insects such as mosquitoes. These are materials, which are not necessarily topically active on their own. Rather they are active in killing or retarding insects. Nonetheless such insecticides are considered TAPIs in accordance with the present invention.

Similarly, materials which are not necessarily topically active, but can be used for prophylaxis such as antiviral, antifungal or antibacterial agents can be considered TAPIs and can be formulated and applied in accordance with the present invention and used to prevent skin being affected by such external forces.

It has been discovered that the use of certain fatty alcohol phosphate esters and mixtures or blends can enhance the activity of certain TAPIs, which are intended to be active in at least one layer of the skin. It has been found that these fatty acid phosphate esters are added to various known or novel topical pharmaceutical preparations, preferably those including an oil-phase or oily materials such as oil-in-water emulsions, oil-in-oil systems, water-in-oil emulsions, ointments and the like, the skin retention time can be increased. These mixed fatty alcohol phosphate ester mixtures may be added to currently available topical preparation containing an oil phase or, at least, oily materials, as that term is known in the personal care industry (e.g., fatty acids, fatty alcohols, waxes, mineral oils, silicone oils and the like) or to a non-oily material.

The use of fatty alcohol phosphate esters in accordance with the present invention is by no means restricted to existing topical pharmaceutical products. Indeed, brand new products can be designed predicated, in whole or in part, on the discovery that these phosphate esters can be used to reduce the rate of transport (reduce flux or increase skin retention time) of a TAPI across the skin. Such new formulations can include, without limitation, new delivery formats for an existing topical product, as well as topical delivery products for a TAPI, which had not previously been formulated in any topically applied product.

The mixed fatty alcohol phosphate ester useful in accordance with the present invention include, amongst others, those described in U.S. Pat. No. 6,117,915, issued to Pereira et al. on Sep. 12, 2000, and assigned on its face to Croda, Inc., the text of which is hereby incorporated by reference. All are mixtures of at least one alkoxylated and at least one nonalkoxylated fatty alcohol phosphate ester. Indeed, in one embodiment, the fatty alcohol phosphate esters of the invention include a mixture containing between about 10% and about 70% by weight of a blend of mono- and di-ester phosphates of alkoxylated fatty alcohols containing between about 12 and 22 carbon atoms and alkoxylated with between about 1 and about 50 moles of an alkylene oxide consisting of ethylene oxide, wherein the mono- and di-ester ratio is between about 10:90 and about 90:10; and between about 90% and about 30% by weight of a blend of mono- and di-ester phosphates of nonalkoxylated fatty alcohols containing between about 12 and 22 carbon atoms, wherein the mono- and di-ester ratio is between about 10:90 and about 90:10.

The fatty alcohol phosphate esters useful in accordance with the present invention include a blend of mono- and di-ester phosphates of alkoxylated and non-alkoxylated fatty alcohols containing between 12 and 22 carbon atoms. Preferred fatty alcohols contain between 14 and 20 carbon atoms. Most preferably, a fatty alcohol blend known as cetearyl alcohol is employed, which is a blend of cetyl and stearyl alcohols, which contain 16 and 18 carbon atoms, respectively.

The phosphate esters of the alkoxylated and non-alkoxylated fatty alcohols of the present invention may be formed by reacting alkoxylated and non-alkoxylated fatty alcohols, respectively, with phosphorous pentoxide (P₂O₅). The alkoxylated fatty alcohols preferably have between about 2 and about 20 moles of the alkoxylating moieties present for each fatty alcohol moiety and are preferably either polyethoxylated, polypropoxylated or both polyethoxylated and polypropoxylated. Therefore, preferred alkoxylated fatty alcohols for use in accordance with the present invention have the structural formula of Formula I: 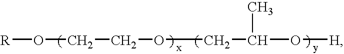
wherein R is a saturated or unsaturated, substituted or unsubstituted fatty moiety containing from 12 to 22 carbon atoms. X and Y are independently zero or integers from 1 to 50, inclusive, and the sum of X and Y is between 1 and 50, inclusive.

The non-alkoxylated fatty alcohols suitable for use in accordance with the present invention have the structural formula of Formula II:
R—OH (II)
R is the same as described above with respect to Formula I.

As is well understood by those of ordinary skill in the art, fatty alcohols [can be derived] are derived from fatty acids, and for this reason, groups such as R are defined as fatty moieties. Fatty alcohols are often commercially prepared from a mixture of fatty acids and contain a mixture of fatty moieties. Therefore, in accordance with the present invention, R may represent a blend of fatty moieties.

Saturated, unsubstituted fatty moieties containing from 14 to 20 carbon atoms are preferred, and, as noted above, a 16 and 18 carbon atom fatty moiety blend, known as a cetearyl blend, is most preferred.

The alkoxylated fatty alcohol depicted in Formula I is prepared by the alkoxylation of the fatty alcohol of Formula II. In the above-depicted alkoxylated fatty alcohol of Formula I, X and Y are preferably independently selected from integers from 2 to 20, inclusive, with the sum of X and Y preferably being between 2 and 20, inclusive.

The alkoxylated fatty alcohols of Formula I are prepared by initially reacting, either sequentially, or in their mixed forms, the fatty alcohols of Formula II with an epoxide, preferably ethylene oxide, propylene oxide, or mixtures thereof, in the presence of an acidic or basic catalyst. It is typical of propylene oxide to branch upon opening of the epoxide ring. Catalysts suitable for this reaction are well-known in the art and include, for example, organic and inorganic alkalies such as alkali metal oxides and hydroxides, e.g., potassium hydroxide, sodium methoxide, sodium borohydride, protic and Lewis acids, e.g., boron trifluoride, stannic chloride and sulfuric acid. Amines, quaternary ammonium compounds, water and other acids may also be employed. Mixtures of catalysts may also be employed. Certain reactive substrates known in the art, for example, acetylenic alkanols may eliminate the need for such catalysts.

Preferably, a basic catalyst is used in this reaction and most preferably from about 0.1 to about 2.0 weight % of potassium or sodium hydroxide, sodium methoxide, sodium borohydride or mixtures thereof, based on the weight of the fatty alcohol. The reaction is carried out under anhydrous conditions to avoid formation of by-products, and at a temperature, which is preferably in the range of from about 110° C. to about 200° C., although higher temperatures may be utilized.

The reaction can be carried out at substantially atmospheric pressure, although it is preferably carried out in an autoclave at pressures of from about 10 psig to about 80 psig. The amount of ethylene oxide or propylene oxide introduced to the reaction zone, and the duration of reaction time, determines the numbers of moles of such components added to the fatty alcohol of Formula II, as is well known by those of ordinary skill in the art. In Formula I, X represents the number of moles of ethylene oxide, which are incorporated into each alkoxylated fatty alcohol chain. Likewise, Y represents the number of moles of propylene oxide that are incorporated into the alkoxylated fatty alcohol chain. As will be readily appreciated by those of ordinary skill in the art, stoichiometric quantities of fatty alcohols, ethylene oxide and propylene oxide are reacted together, and stoichiometric quantities of the alkoxylated fatty alcohol and P₂O₅ are then reacted together to form the mono- and di-phosphate ester alkoxylated fatty alcohol blend.

For alkoxylation reactions in which the fatty alcohol is both ethoxylated and propoxylated, that is, when neither X nor Y is zero, the alkoxylation reaction is preferably carried out sequentially in that the fatty alcohol is first reacted with the propylene oxide and after complete reaction, the ethylene oxide is introduced into the reaction. After complete reaction of the ethylene oxide, an acid, e.g., phosphoric acid or acetic acid, is introduced into the reaction mixture to neutralize the basic catalyst.

The fatty acid phosphate ester mixtures of the present invention, in addition to being a blend of alkoxylated and non-alkoxylated fatty alcohol phosphate esters, are also mono- and diester phosphate blends of both the alkoxylated and non-alkoxylated fatty alcohol phosphate esters. Thus, the alkoxylated fatty alcohol of Formula I, prepared as described above, is next reacted in a conventional phosphating reaction with P₂O₅ to form a mono- and diester phosphate alkoxylated fatty alcohol blend. The fatty alcohol phosphate esters can also be prepared by reacting P₂O₅ with mixtures of nonalkoxylated fatty alcohols and alkoxylated fatty alcohols.

The phosphating reaction is typically performed by combining stoichiometric quantities of the alkoxylated fatty alcohol and the P₂O₅. As is well understood by those of ordinary skill in the art, the ratio of the two reagents will depend upon the ratio of mono- and diester phosphates desired. To obtain significant quantities of diester in the first place, a stoichiometric excess of P₂O₅ should be employed, with greater excess levels of P₂O₅ employed to increase the level of diester obtained. A 1:3 molar ratio of P₂O₅ to alkoxylated fatty alcohol is preferred.

The alkoxylated fatty alcohol is heated to a temperature between about 35° C. and about 90° C., and preferably at a temperature between about 50° C. and about 80° C., and then combined with mixing with P₂O₅ to form a reaction mixture. The alkoxylated fatty alcohol is a liquid at this temperature; therefore, a reaction solvent is not needed. The reaction is then allowed to continue until essentially complete, typically until about 10% or less of unreacted alkoxylated fatty alcohol and trace amounts of unreacted P₂O₅, now in the form of phosphoric acid, remain, usually about four hours. The reaction mixture is then recovered as a mono- and diester phosphate blend of alkoxylated fatty alcohols.

The alkoxylated fatty alcohol phosphate esters are then combined with a mono- and diester phosphate blend of non-alkoxylated fatty alcohols. The phosphate ester blend of non-alkoxylated fatty alcohols is prepared essentially the same as the phosphate ester blend of the alkoxylated fatty alcohols, by reacting stoichiometric quantities of the fatty alcohol of Formula II and P₂O₅ essentially in the same manner as described above for the alkoxylated fatty alcohol phosphate ester blend.

As noted above, mixed forms of fatty alcohols containing from 12 to 22 carbon atoms can be employed. Therefore, the resulting phosphate ester blends of alkoxylated and non-alkoxylated fatty alcohols can contain mixtures of alkoxylated and non-alkoxylated fatty alcohol phosphate esters containing from 12 to 22 carbon atoms.

The phosphate ester compositions of the present invention are then prepared by blending the mono- and di-phosphate ester blends of alkoxylated fatty alcohols with the mono- and diester phosphate blends of non-alkoxylated fatty alcohols. Quantities of the alkoxylated and non-alkoxylated phosphate esters are added to a stirred vessel and heated with mixing at a temperature between about 60° C. and about 90° C., and preferably at a temperature between 75° C. and 85° C., until a uniform homogeneous mixture is obtained, typically about 30 minutes.

The amount of alkoxylated fatty alcohol phosphate esters blended with non-alkoxylated fatty alcohol phosphate esters will depend upon the ultimate ratio of phosphate esters of alkoxylated and non-alkoxylated fatty alcohols desired. The emulsifier compositions of the present invention contain between about 10% and about 90% of alkoxylated fatty alcohol phosphate esters and between about 90% and about 10% of non-alkoxylated fatty alcohol phosphate esters. Preferred emulsifier compositions contain the ratio of alkoxylated fatty alcohol phosphate esters to non-alkoxylated fatty alcohol phosphate esters between about 20:80 and about 80:20, and more preferably between about 30:70 and about 70:30. The desired ratio is obtained by combining the alkoxylated fatty alcohol phosphate esters and non-alkoxylated fatty alcohol phosphate esters on a weight ratio basis.

The fatty alcohol phosphate ester mixtures of the present invention may be formulated as emulsifying waxes. Emulsifying waxes are essentially a blend of the emulsifier compositions of the present invention with a fatty alcohol containing from 12 to 22 carbon atoms. Other mixed alkoxylated and nonalkoxylated phosphate esters can be selected from Oleth-5 Phosphate and Dioleyl Phosphate, Oleth-3 Phosphate, DEA Oleth-3 Phosphate, Oleth-10 Phosphate, DEA-Oleth-10 Phosphate. In one embodiment, the mixed phosphate esters, and indeed the other ingredients used in the formulations, are liquid at room temperature. In a particularly preferred embodiment, the final product is one used for veterinary applications where the material is applied to a single place on the animal's skin and it spreads to cover much, if not all, of the animal's skin surface.

Oil-in-water emulsions typically contain fatty alcohol thickening agents, and fatty alcohol based emulsifying waxes represent a convenient form by which fatty alcohols may be added to oil-in-water emulsions in combination with an appropriate quantity of emulsifier. Thus, the amount of the composition of the present invention combined with a fatty alcohol to form an emulsifying “wax” will depend upon the ratio of fatty alcohol to emulsifier in the oil-in-water emulsion to be prepared. Therefore, emulsifying waxes in accordance with the present invention may contain from about 5% to about 90% by weight of the emulsifier composition of the present invention, although preferred emulsifying waxes will contain up to about 30% by weight of the emulsifier composition of the present invention.

Preferred emulsifying waxes in accordance with the present invention will be based upon one or more fatty alcohols containing from 14 to 20 carbon atoms. The cetearyl alcohol blend of 16 and 18 carbon atom fatty alcohols is most preferred.

Oil-in-water emulsions in accordance with the present invention are a preferred form of delivery vehicle for TAPIs and are generally made by combining an oil phase, a water phase and an amount of an emulsifier effective to form an emulsion of the oil and water phase. Likewise, oil-in-water microemulsions in accordance with the present invention combine an oil phase, a water phase and an amount of an emulsifier effective to form a microemulsion of the oil and water phases. The fatty alcohol phosphate ester mixtures may be used as the emulsifier. However, any other emulsifier may be used to build the emulsion, as the primary roles of the fatty alcohol phosphate esters of the invention are believed to be permeation retardation. Any activity as an emulsifier is a bonus, although it is understood that the fatty alcohol phosphate ester mixtures described herein are excellent emulsifiers. Other emulsifiers that may be used include polysorbates and ethoxylated fatty alcohols. Accordingly, no other emulsifier may be necessary.

Typical emulsions contain an oil phase at a level between about 2% and about 80% by weight, preferably between about 5% and about 60% by weight, and more preferably between about 15% and about 40% by weight; and a water phase at a level between about 10% and about 98% by weight, preferably between about 20% and about 80% by weight, and more preferably between about 40% and about 70% by weight, based on the total emulsion weight.

For microemulsions, significantly higher levels of emulsifier are often used, so that the oil droplets formed are so small that the emulsion is transparent. Typically, the emulsifier is present at a level greater than or equal to that of the oil phase up to a level of about 300% by weight of the oil phase. A level of between about 150% and about 275% by weight of the oil phase is preferred, with a level of between about 225% and about 250% of the oil phase being more preferred. Such microemulsions typically contain an oil phase at a level of between about 5% and about 80% by weight, and preferably between about 20% and about 40% by weight. The water phase is typically at a level between about 20% and about 95% by weight, preferably between about 30% and about 70% by weight, and most preferably between about 40% and about 60% based on the total weight of the microemulsion.

The oil-in-water emulsions of the present invention are formulated utilizing techniques that are well-known in the art. Typically, all water-soluble ingredients are mixed together to form the water phase and all water-insoluble ingredients are mixed together to form the oil phase. The two phases are then combined with the emulsifier composition of the present invention and mixed until an emulsion is formed.

The microemulsion compositions of the present invention are formulated in a similar manner, particularly as described in U.S. patent application Ser. No. 08/052,557, filed Apr. 23, 1993, now abandoned, the disclosure of which is incorporated herein by reference. The emulsifier compositions, the mixed phosphate esters, of the present invention are substituted for the surface active agents described in that application.

There are many methods for manufacturing products as microemulsions. In one method, all of the ingredients are charged to a reactor and heated, with stirring, until a homogeneous mixture is achieved. Generally, the formulation is heated to between about 80-85° C. and then cooled to about 35° C. or less. During cooling, the microemulsion is established.

One preferred method of making microemulsions in accordance with the present invention requires the separate and discrete formation of a water phase and an oil phase. In some cases, the water phase may be composed of just water. In other cases, the water phase may include water and the mixed phosphate esters of the invention.

The oil phase includes at least one oil, preferably mineral oil, and at least one of the mixed phosphate esters of the invention.

The emulsion and microemulsion based topical preparations of the invention are formulated utilizing techniques that are well-known in the art. Typically, the water-soluble ingredients are dissolved in the water-phase and the water-insoluble ingredients are combined with the oil phase prior to formation of the emulsion. Typically, the ingredients are combined with mixing and the addition of heat if necessary until uniform, homogeneous phases are formed. The two phases are then combined with the addition of the emulsifier composition of the present invention to form an emulsion or microemulsion based topical preparation.

Those of ordinary skill in the art can readily identify whether a particular active agent is water-soluble or water-insoluble and therefore whether it should be included in the water phase or oil phase of the emulsion. Likewise, whether the topical preparation will be based on an emulsion, microemulsion, ointment or other delivery format is more or less an aesthetic determination based upon whether a milky, opaque product is desired, or whether a clear gel-like microemulsion is preferred. In selecting the microemulsion product form, potential skin irritation from the use of elevated levels of emulsifier and/or surfactant should be considered.

The topical preparations of the present invention, in addition to including one or more active ingredients (API and/or TAPI) in an oil-in-water, oil-in-oil or water-in-oil emulsion or microemulsion may also include coloring agents, fragrances, proteins, salts, preservatives, essential oils, antiacne agents, and the like. These additional components may be added in various amounts as is well-known in the cosmetic and personal care product formulation art. Such ingredients need not be added prior to the emulsion formation, but may instead be combined with the emulsion with mixing and the addition of heat if necessary until a uniform, homogeneous product is formed.

While described in terms of oil-in-water emulsion and in mixtures of fatty alcohol ester and emulsified waxes, neither is essential. The fatty alcohol phosphate ester mixtures of the present invention can be added directly to an already existing formulation and need not be mixed with a fatty alcohol based wax or other wax material. In addition, the formulations in accordance with the present invention need not be oil-in-water emulsions. They can be oil-in-oil, water-in-oil emulsions, mixtures and nonemulsified systems and preparations based on at least one oily component as that term is understood in the personal care industry.

While emulsions and particularly oil-in-oil and oil-in-water emulsions and indeed water-in-oil emulsions are a preferred aspect of the present invention, they are not the only aspect of the invention. Transdermal and topical products and methods as described herein can be accomplished using creams, ointments, gels, pastes and the like, as long as they meet the criteria of the present invention in terms of providing a decrease in flux and/or a concomitant increase in skin retention time. Nonemulsified vehicles which can be used include, without limitation, Triglycerides Oils, e.g. Sesame Oil, Soybean Oil, etc., Ethyl Oleate, Oleic Acid, Cetearyl Ethylhexanoate, Caprylic-Caparic Triglyceride, and PPG-2 Myristyl Ether Propionate. Therefore, products in accordance with the present invention include emulsions as well as creams, ointments, gels, lotions and pastes.

The total amount of mixed fatty alcohol phosphate esters used in a formulation in accordance with the present invention will vary with the number of factors, including, amongst other things, the remaining ingredients of the formulation, the TAPI or API to be delivered, the degree to which the transport of the TAPI or API across the skin is to be retarded or delayed and the composition of the fatty alcohol phosphate mixture itself. However, at a minimum, the amount used should be an amount sufficient to provide at least some measurable amount of improvement in the flux or skin retention time of some amount of the TAPI or API when compared to an identical formulation without the fatty alcohol phosphate esters. And again, such an improvement in flux or skin retention time may be measured by assay of the actual active or by adding a surrogate or proxy such as ³H-cortisol and measuring the flux or skin retention time of the surrogate.

In one embodiment, the total amount of mixed fatty alcohol phosphate esters in accordance with the present invention, based on the total amount of phosphate containing active species, is provided an amount of at least about 0.05% by weight of the total formulation. The upper limit is not critical, however, a point of diminishing returns may be reached. However generally, the amount may range from between about 0.1% to about 20% by weight of the total formulation, more preferably between about 0.5% and about 10% by weight and most preferably between about 0.5% and about 5% by weight of the total formulation.

One particularly preferred material, which may be used, is sold under the name CRODAFOS CES available from Croda, Inc., 300-A Columbus Circle, Edison, N.J. 08837. CRODAFOS CES is a mixture of cetearyl alcohol and dicetyl phosphate and ceteth-10 phosphate. The amount of phosphate containing fatty alcohol species is roughly 25% with a fatty alcohol wax comprising the other 75% by weight thereof. Therefore, the use of 3% of CRODAFOS CES will provide approximately 0.75 weight percent fatty alcohol phosphate esters based on total weight of the final formulation. Other mixed phosphate esters may be selected from mixtures of, for example, Cetearyl Alcohol and Ceteth-20 Phosphate and Dicetyl Phosphate, Oleth-5 Phosphate and Dioleyl Phosphate, Oleth-3 Phosphate, DEA Oleth-3 Phosphate, Oleth-10 Phosphate, DEA-Oleth-10 Phosphate, PPG-5 Ceteth-10 Phosphate, Cetyl Phosphate and Stearic Acid.

Any topically active pharmaceutical ingredient (“TAPI”) may, potentially, be delivered in a formulation in accordance with the present invention. Preferably these materials will be active in or on the skin, and on conditions that affect the skin. In the case of water-in-oil emulsions, such TAPIs must also be capable of existing in a system which is both aqueous and nonaqueous without degradation, loss of potency, discoloration or the like.

Nontopically active pharmaceutical ingredient, also referred to as “APIs,” are pharmaceuticals, drugs or other active materials which are intended to be administered into the blood stream where they exert their influence on the body. Though these APIs are identified as being nontopically active, they may indeed be materials which can exert topical activity. However, the reason for their administration in this instance is not for the treatment of a topical condition. Alternatively, these may be used for the treatment of a topical condition by something other than topical administration. For example, certain steroids may be useful to treat topical conditions. Steroids may also be used to treat other conditions within the body. If the steroids are administered to an area of a patient's skin afflicted by a particular condition, bacterial infection, fungal infection, or the like, for the purposes of treating that condition then the steroid would be considered a TAPI. However, if it were administered through nonafflicted skin, with the intention that the steroid exerts its activity within the body, it would be considered an API.

Similarly, there are skin conditions, which can affect broad areas of a patient's surface area, which are treated by drugs, which are currently ingested orally. These drugs can now be administered through the skin in accordance with the present invention and, as they are intended to treat a skin condition, they would be considered APIs in the context of the invention, rather than TAPIs.

Active ingredients, both those to be applied and active in or on the skin, nail, or other topical surfaces (TAPIs) and those which are to be delivered through the skin to the circulatory system (APIs) in accordance with the present invention can be any pharmaceutically active ingredient including, without limitation, abortifacient/interceptive, ace-inhibitor, α-adrenergic agonist, β-adrenergic agonist, α-adrenergic blocker, β-adrenergic blocker, adrenocortical steroid, adrenocortical suppressant, adrenocorticotropic hormone, alcohol deterrent, aldose reductase inhibitor, aldosterone antagonist, 5-alpha reductase inhibitor, anabolic, analeptic, analgesic, androgen, angiotensin converting enzyme inhibitor, angiotensin II receptor antagonist, anorexic, antacid, anthelmintic, antiacne, antiallergic, antialopecia agent, antiamebic, antiandrogen, antianginal, antiarrhythmic, antiarteriosclerotic, antiarthritic/antirheumatic, antiasthmatic, antibacterial, antibacterial adjuncts, antibiotic, anticancer, anticholelithogenic, anticholesteremic, anticholinergic, anticoagulant, anticonvulsant, antidepressant, antidiabetic, antidiarrheal, antidiuretic, antidote, antidyskinetic, antieczematic, antiemetic, antiepileptic, antiestrogen, antifibrotic, antiflatulent, antifungal, antiglaucoma, antigonadotropin, antigout, antihemorrhagic, antihistaminic, antihypercholesterolemic, antihyperlipidemic, antihyperlipoproteinemic, antihyperphosphatemic, antihypertensive, antihyperthyroid, antihypotensive, antihypothyroid, anti-infective, anti-inflammatory, antileprotic, antileukemic, antilipemic, antimalarial, antimanic, antimethemoglobinemic, antimigraine, antimycotic, antinauseant, antineoplastic, antineoplastic adjunct, antineutropenic, antiosteoporotic, antipagetic, antiparkinsonian, antiperistaltic, antipheochromocytoma, antipneumocystis, antiprostatic hypertrophy, antiprotozoal, antipruritic, antipsoriatic, antipsychotic, antipyretic, antirheumatic, antirickettsial, antiseborrheic, antiseptic/disinfectant, antispasmodic, antisyphilitic, antithrombocythemic, antithrombotic, antitubercular, antitumor, antitussive, antiulcerative, antiurolithic, antivenin, antivertigo, antiviral, anxiolytic, aromatase inhibitors, astringent, benzodiazepine antagonist, beta-blocker, bone resorption inhibitor, bradycardic agent, bradykinin antagonist, bronchodilator, calcium channel blocker, calcium regulator, calcium supplement, cancer chemotherapy, capillary protectant, carbonic anhydrase inhibitor, cardiac depressant, cardiotonic, cathartic, CCK antagonist, central stimulant, cerebral vasodilator, chelating agent, cholecystokinin antagonist, cholelitholytic agent, choleretic, cholinergic, cholinesterase inhibitor, cholinesterase reactivator, CNS stimulant, cognition activator, contraceptive, control of intraocular pressure, converting enzyme inhibitor, coronary vasodilator, cytoprotectant, debriding agent, decongestant, depigmentor, dermatitis herpetiformis suppressant, diagnostic aid, digestive aid, diuretic, dopamine receptor agonist, dopamine receptor antagonist, ectoparasiticide, emetic, enkephalinase inhibitor, enzyme, enzyme cofactor, enzyme inducer, estrogen, estrogen antagonist, expectorant, fibrinogen receptor antagonist, gastric and pancreatic secretion stimulant, gastric proton pump inhibitor, gastric secretion inhibitor, gastroprokinetic, glucocorticoid, α-glucosidase inhibitor, gonad-stimulating principle, gout suppressant, growth hormone inhibitor, growth hormone releasing factor, growth stimulant, hematinic, hematopoietic, hemolytic, hemostatic, heparin antagonist, hepatoprotectant, histamine H₁-receptor antagonist, histamine H₂-receptor antagonist, HIV proteinase inhibitor, HMG CoA reductase inhibitor, hypnotic, hypocholesteremic, hypolipidemic, hopotensive, immunomodulator, immunosuppressant, intropic agent, insulin sensitizer, ion exchange resin, keratolytic, lactation stimulating hormone, laxative/cathartic, leukotriene antagonist, LH-RH agonist, lipotropic, 5-lipoxygenase inhibitor, lupus erythematosus suppressant, major tranquilizer, matrix metalloproteinase inhibitor, mineralocorticoid, minor tranquilizer, miotic, monoamine oxidase inhibitor, mucolytic, muscle relaxant, mydriatic, narcotic analgesic, narcotic antagonist, nasal decongestant, neuroleptic, neuromuscular blocking agent, neuroprotective, nootropic, nsaid, opioid analgesic, oral contraceptive, ovarian hormone, oxytocic, parasympathomimetic, pediculicide, pepsin inhibitor, peripheral vasodilator, peristaltic stimulant, pigmentation agent, plasma volume expander, potassium channel activator/opener, pressor agent, progestogen, prolactin inhibitor, prostaglandin/prostaglandin analog, protease inhibitor, proton pump inhibitor, pulmonary surfactant, 5α-reductase inhibitor, replenishers/supplements, respiratory stimulant, retroviral protease inhibitor, reverse transcriptase inhibitor, scabicide, sclerosing agent, sedative/hypnotic, serenic, serotonin noradrenaline reuptake inhibitor, serotonin receptor agonist, seratonin receptor antagonist, serotonin uptake inhibitor, skeletal muscle relaxant, somatostatin analog, spasmolytic, stool softener, succinylcholine synergist, sympathomimetic, thrombolytic, thromboxane A₂-receptor antagonist, thromboxane A₂-sythetase inhibitor, thyroid hormone, thyroid inhibitor, thyrotropic hormone, tocolytic, topical protectant, topoisomerase I inhibitor, topoisomerase II inhibitor, tranquilizer, ultraviolet screen, uricosuric, vasodilator, vasopressor, vasoprotectant, vitamin/vitamin source, vulnerary, Wilson's disease treatment, xanthine oxidase inhibitor. Preferably, the drug is selected from the group consisting of acyclovir; auranofin; bretylium; cytarabine; doxepin; doxorubicin; hydralazine; ketamine; labetalol; mercaptopurine; methyldopa; nalbuphine; nalozone; pentoxifyll; pyridostigmine; terbutaline; verapamil; buserelin; calcitonin; cyclosporin; oxytocin and heparin. Also encompassed by the terms TAPI and API are the drugs and pharmaceutical active ingredients described in Mantelle U.S. Pat. No. 5,234,957 includes 18 through 21. The terms Topically Active Pharmaceutical Ingredient(s) and TAPI and Active Pharmaceutical Ingredients(s) and API do not include the actives described in U.S. Pat. No. 6,117,915 which include: UV absorbing agents, aqueous moisturizing agents, oily moisturizing agents, film-forming polymers, thickening agents, secondary emulsifiers other than said mono- and diester phosphates of said alkoxylated and non-alkoxylated fatty alcohols, antiseptic agents, skin conditioning agents, hair conditioning agents, deodorant actives, humectants, rheological modifiers, the above-mentioned protein reducing agents or protein hydrolyzing agents for permanent wave and hair relaxer products, and the like. Some of these, however, may be present in formulations in accordance with the present invention as additional ingredients, additional actives, and/or excipients. For example, topical formulations could include a nonsteroidal anti-inflammatory (“NSAID”) agent as a TAPI and a UV absorbing agent may be present as an additional active.

Particularly preferred TAPIs and APIs include one or more cyclic or aromatic groups and/or bulky molecules with considerable steric hindrance. These actives include both prescription and over the counter actives, as well as vitamins, collagen, insect repellents, bioflavonoids, as well as products based on squalene, salicylic acid, resouncinol, miconazole, DEET (N,N, diethyl-m-toluamide), tocopherol, tocopherol acetate, retinoic acid, retinol, and retinoids. Examples of suitable anti-acne medicaments include sulfur, erythromycin, zinc, and benzoyl peroxide. Other desirable TAPIs and APIs also include, without limitation, compounds based on perhydrocyclopentanophenanthrene nucleus, the nucleus of steroids, cholesterol and lanosterol, as well as derivatives thereof. Derivatives include, without limitation, androgens such as testosterone and derivatives thereof such as, without limitation, dihydrotesterone, progestational hormones such as progesterone and derivatives thereof, and corticosteroids such as cortisols (hydrocortisones), corticosterone and derivatives thereof. Estrogen based compounds such as beta-Estradiol and derivatives thereof are also desirable. Birth control substances, nicotine and nitroglycerine may also be used.

In general, the predetermined amount of active ingredient incorporated into each formulation may be selected according to known principles of pharmacy. “Formulation” means an amount of active ingredient and pharmaceutically acceptable excipients combined together which are ultimately incorporated into an overall dosage form. Generally, the amount of active ingredient incorporated is a pharmaceutically effective amount. A “pharmaceutically effective amount” is the amount or quantity of an active ingredient which is sufficient to elicit the required or desired therapeutic response. In other words, it is the amount which is sufficient to elicit an appreciable biological response when administered to a patient. Of course, the amount of active ingredient used can vary greatly. It depends on the size of the dosage, the requirements of other ingredients, the size, age, weight, sex, condition of the patient, their medical condition, and the number of, for example, tablets which constitute a single dose. Typically, an active ingredient in each dose can be present in an amount of from about 0.1 mg to about 1000 mg, preferably from about 1 mg to about 500 mg and more preferably from about 4 mg to about 200 mg. Conventional amounts of pharmaceutically acceptable excipients can be used in this these formulations as well.

The amount of TAPI or API to be provided in accordance with the present invention will vary with the TAPI or API, the condition of the patient, the length and duration of dosing, the sound judgment of treating professionals, and the Food and Drug Administration or other related regulatory agencies, the solubility or compatibility of the active in the formulation and the like. It will also vary with the condition being treated and whether or not the invention is being used to treat a topical condition or a condition where delivery of the API is through the blood stream. However, generally, formulations in accordance with the present invention will contain at least about 0.1% TAPI or API by weight based on the weight of the total formulation. More preferably, the amount of TAPI or API by weight in the formulation will range from between about 0.1% to about 50%, more preferably from about 0.1% to about 10%, and most preferably from about 0.1% to about 5%. The remainder is excipients, additional ingredients, and/or carriers.

The compositions of the invention may also include a wide range of miscellaneous ingredients (also known as carriers, excipients, or additional ingredients). Some suitable miscellaneous ingredients commonly used in the cosmetic and personal care industry are described in The CTFA Cosmetic Ingredient Handbook, (2nd Ed., 1992), which is incorporated by reference herein.

Thus, the compositions of the invention may also include one or more absorbents, anti-acne agents, antiperspirants, anti-caking agents, antifoaming agents, antimicrobial agents, antioxidants, antidandruff agents, astringents, binders, buffers, biological additives, buffering agents, bulking agents, chelating agents, chemical additives, coupling agents, conditioners, colorants, cosmetic astringents, cosmetic biocides, denaturants, drug astringents, detergents, dispersants, external analgesics, film formers, foaming agents, fragrance components, humectants, keratolytics, opacifying agents, pH adjusters, preservatives, propellants, proteins, retinoids, reducing agents, sequestrants, skin bleaching agents, skin-conditioning agents (humectants, miscellaneous, and occlusive), skin soothing agents, skin healing agents, softeners, solubilizing agents, lubricants, penetrants, plasticizers, solvents and co-solvents, salts, essential oils, and vitamins. These may all be present in amounts conventionally used for such ingredients in the topical pharmaceutical, personal care and cosmetics industries, and can range from as little as about 0.01% to about 60% by weight, more preferably from about 0.5% to about 30% by weight. As noted previously, preferred embodiments in accordance with the present invention are provided in the form of an emulsion. Thus, the vehicle used for the mixed fatty alcohol phosphate esters and the TAPI or API is an emulsion. These can include oil-in-water, water-in-oil, and oil-in-oil emulsions. Nonemulsified vehicles including solvents and co-solvents or other carriers may also be used as discussed herein. In particular, topical preparations in accordance with the present invention preferably include an emollient, an emulsifier, a thickener, water, a preservative, a stabilizer, a pH-adjusting substance, a color, a solvent, a co-solvent, a dispersion aide or a solid particulate. The topical pharmaceutical preparations of the present invention can be provided in any known form. However, preferred are creams, milks, lotions, gels, salves, ointments, sprays, mousses, liquids, and sticks. In addition, the topical preparations of the present invention can be applied and then covered with a bandage, or patch, or some other occlusive barrier, or may be provided as part of a pre-made, ready-to-use topical device, such as a bandage, pad, patch or the like. Thus, the material may be applied to a gauze, pad, swab, cotton ball, batting, bandage, patch or occlusive barrier. In one particular embodiment, a preparation in accordance with the present invention can be provided in a well or reservoir or as part of a unitary adhesive or nonadhesive mixture. This material can be sandwiched between a peelable or removable layer and a backing layer, which often forms the reservoir, which is occlusive. While these sorts of patch structures are typically useful for transferal drug applications, they can be used for the topical preparations of the present invention which provide enhanced topical exposure.

The compositions of the invention may also include one or more emollient compounds such as fats, waxes, lipids, silicones, hydrocarbons, fatty alcohols and a wide variety of solvent materials. The amount of the emollient depends on the application. For the final product compositions, emollients are included in the amount of up to 50% by weight of the composition, preferably, from about 0.1% to about 20%, and more preferably, from about 0.5% to about 10% by weight of the composition.

Examples of suitable emollients include C₈₋₃₀ alkyl esters of C₈₋₃₀ carboxylic acids; C₁₋₆ diol monoesters and diesters of C₈₋₃₀ carboxylic acids; monoglycerides, diglycerides, and triglycerides of C₈₋₃₀ carboxylic acids, cholesterol esters of C₈₋₃₀ carboxylic acids, cholesterol, and hydrocarbons. Examples of these materials include diisopropyl adipate, isopropyl myristate, isopropyl palmitate, ethylhexyl palmitate, isodecyl neopentanoate, C₁₂₋₁₅ alcohols benzoates, diethylhexyl maleate, PPG-14 butyl ether, PPG-2 myristyl ether propionate, cetyl ricinoleate, cholesterol stearate, cholesterol isosterate, cholesterol acetate, jojoba oil, cocoa butter, shea butter, lanolin, lanolin esters, mineral oil, petrolatum, and straight and branched C₁₆-C₃₀ hydrocarbons.

Also useful are straight and branched chain fatty C₈-C₃₀ alcohols, for example, stearyl alcohol, isostearyl alcohol, phenyl alcohol, cetyl alcohol, isocetyl alcohol, and mixtures thereof. Examples of other suitable emollients are disclosed in U.S. Pat. No. 4,919,934; which is incorporated herein by reference in its entirety.

Other suitable emollients are various alkoxylated ethers, diethers, esters, diesters, and triesters. Examples of suitable alkoxylated ethers include PPG-10 butyl ether, PPG-11 butyl ether, PPG-12 butyl ether, PPG-13 butyl ether, PPG-14 butyl ether, PPG-15 butyl ether, PPG-16 butyl ether, PPG-17 butyl ether, PPG-18 butyl ether, PPG-19 butyl ether, PPG-20 butyl ether, PPG-22 butyl ether, PPG-24 butyl ether, PPG-30 butyl ether, PPG-11 stearyl ether, PPG-15 stearyl ether, PPG-10 oleyl ether, PPG-7 lauryl ether, PPG-30 isocetyl ether, PPG-10 glyceryl ether, PPG-15 glyceryl ether, PPG-10 butyleneglycol ether, PPG-15 butylene glycol ether, PPG-27 glyceryl ether, PPG-30 cetyl ether, PPG-28 cetyl ether, PPG-10 cetyl ether, PPG-10 hexylene glycol ether, PPG-15 hexylene glycol ether, PPG-10 1,2,6-hexanetriol ether, PPG-15 1,2,6-hexanetriol ether, and mixtures thereof.

Examples of alkoxylated diethers include PPG-10 1,4-butanediol diether, PPG-12 1,4-butanediol diether, PPG-14 1,4-butanediol diether, PPG-2 butanediol diether, PPG-10 1,6-hexanediol diether, PPG-12 1,6-hexanediol diether, PPG-14 hexanediol diether, PPG-20 hexanediol diether, and mixtures thereof. Preferred are those selected from the group consisting of PPG-10 1,4-butanediol diether, PPG-12 1,4-butanediol diether, PPG-10 1,6-hexandiol diether, and PPG-12 hexanediol diether, and mixtures thereof.

Examples of suitable alkoxylated diesters and triesters are disclosed in U.S. Pat. Nos. 5,382,377, 5,455,025 and 5,597,555, assigned to Croda Inc., and incorporated herein by reference.

Suitable lipids include C₈-C₂₀ alcohol monosorbitan esters, C₈-C₂₀ alcohol sorbitan diesters, C₈-C₂₀ alcohol sorbitan triesters, C₈-C₂₀ alcohol sucrose monoesters, C₈-C₂₀ alcohol sucrose diesters, C₈-C₂₀ alcohol sucrose triesters, and C₈-C₂₀ fatty alcohol esters of C₂-C₆₂-hydroxy acids. Examples of specific suitable lipids are sorbitan diisostearate, sorbitan dioleate, sorbitan distearate, sorbitan isosotearate, sorbitan laurate, sorbitan oleate, sorbitan palmitate, sorbitan sesquioleate, sorbitan esquistearte, sorbitan stearate, sorbitan triiostearte, sorbitan trioleate, orbitan tristeate, sucrose cocoate, sucrodilaurate, sucrose distearate, sucrose laurate, sucrose myristate, sucrose oleate, sucrose palmitate, sucrose ricinoleate, sucrose stearate, sucrose tribehenate, sucrose tristearate, myristyl lactate, stearyl lactate, isostearyl lactate, cetyl lactate, palmityl lactate, cocoyl lactate, and mixtures thereof.

Other suitable emollients include mineral oil, petrolatum, cholesterol, dimethicone, dimethiconol, stearyl alcohol, cetyl alcohol, behenyl alcohol, diisopropyl adipate, isopropyl myristate, myristyl myristate, cetyl ricinoleate, sorbitan distearate, sorbitan dilaurate, sorbitan stearate, sorbitan laurate, sucrose laurate, sucrose dilaurate, sodium isostearyl lactylate, lauryl pidolate, sorbitan stearate, stearyl alcohol, cetyl alcohol, behenyl alcohol, PPG-14 butyl ether, PPG-15 stearyl ether, and mixtures thereof.

Emulsifiers

The compositions of the invention may also include various emulsifiers other than the mixed phosphate esters of the present invention. In the final product compositions of the invention, emulsifiers may be included in the amount of up to about 10%, preferably, in the amount of from about 0.5% to about 5% by weight of the composition. The examples of suitable emulsifiers include stearamidopropyl PG-dimonium chloride phosphate, stearamidopropyl ethyldimonium ethosulfate, stearamidopropyl dimethyl (myristyl acetate) ammonium chloride, stearamidopropyl dimethyl cetearyl ammonium tosylate, stearamidopropyl dimethyl ammonium chloride, stearamidopropyl dimethyl ammonium lactate, polyethyleneglycols, polypropyleneglyocis, and mixtures thereof.

Thickeners

The compositions of the invention may also include various thickeners, such as cross-linked acrylates, nonionic polyacrylamides, xanthan gum, guar gum, gellan gum, and the like; polyalkyl siloxanes, polyaryl siloxanes, and aminosilicones. In the final product compositions of the invention, thickeners may be included in the amount of up to about 10%, preferably, in the amount of from about 0.2% to about 5% by weight of the composition.

Examples of the suitable thickening silicone compounds include polydimethylsiloxane, phenylsilicone, polydiethylsiloxane, and polymethylphenylsiloxane. Some of the suitable silicon compounds are described in European Patent Application EP 95,238 and U.S. Pat. No. 4,185,017, which are incorporated herein by reference. The compositions of the invention may also include silicone polymer materials, which provide both style retention and conditioning benefits to the hair. Such materials are described in U.S. Pat. No. 4,902,499, which is incorporated herein by reference.

Examples of suitable film formers include glycerin/diethylene glycol myrystate copolymer, glycerin/diethylene glycol adipate copolymer, ethyl ester of PVM/MA copolymer, PVP/dimethiconylacrylate/polycarbamyl/polyglycol ester, and mixtures thereof. If the film formers are present in the final product compositions, the amount may vary from about 0.1% to about 15.0% by weight of the composition, preferably, from about 0.1% to about 2.5% by weight of the composition.

Methods of Analysis of Skin Penetration

In one clinical measure of penetration depth is used here only to illustrate the invention, 100 mg of the creams described in Example 1 with or without 2% hydrocortisone were applied to the volar forearm skin for 2 hours uncovered. After 2 hours the volar surface was examined by in vivo confocal laser scanning microscopy using the technique described in in vivo Real-Time Confocal Imaging, J. V. Jester, P. M. Andrews, W. M. Petroll, M. A. Lemp and H. D. Cavanaugh, Jour. Electron Microscopy Techniques, 18:50-60 (1991), in vivo confocal microscopy of human skin: A new design for cosmetology and dermatology, P. Corcuff, G. Gonnord, G. E. Pierard and J. L. Leveque, Scanning: Vol. 18, 351-355 (1996). Images can be collected in real time. 2.1 micron optical sections can be viewed from the top of the stratum corneum to the granulosum ˜14.7 microns depth.

### EXAMPLES

### Example 1

For determining flux and/or skin retention time, the following test may be employed. Human skin from breast reduction surgery was used for experimentation. All skin used was deemed intact but not metabolically active. Prior to experimentation the skin integrity was determined by measuring the migration of tritiated water (Bronaugh, et al. 1986). The formulas used contained mineral oil, cetylstearyl alcohol and emulsifying wax NF with and without the fatty acid phosphate ester of the present invention. Specifically, the first formulation included: 5% Polawax, 5% Mineral Oil, 3% CES, and 1% Germaben II. “CES” refers to CRODAFOS CES described herein which is a mixture of cetylstearyl alcohol (2.25% by weight of the final formulation) and a mixture of alkoxylated and nonalkoxylated fatty acid phosphate esters (0.75% by weight of the final formulation). The second formulation is similar and is composed of 5% Polawax, 5% Mineral Oil, 2.25% Crodacol S-70 (cetylstearyl alcohol 70%) and 1% Germaben II with the balance being water. Crodacol S-70 is cetylstearyl alcohol. Thus the only significant difference between the two is whether or not the formulations contain any mixed fatty acid phosphate esters (“CES” or no “CES”).

Prior to use 11.2 microcuries (uCi) of [1,2,6,7−³H] cortisol was added to the prewarmed (37° C.) formulas as noted above, mixed thoroughly, and incubated at 370C. Split-thickness skin was prepared by dermatoming to an approximate thickness of 0.28 mm. Franz cell finite dosing chambers were used, tissue was placed dermis side down onto phosphate buffered saline (PBS), and 100 microliters of the formulas were placed directly in contact with the stratum corneum. PBS was removed at predetermined times and the radioactivity counted. The lotions were removed and the tissue digested with 50% H₂O₂ and then counted. Experiments were done in duplicate with each condition tested in triplicate.

³H cortisol was initially applied for 6 hours. There was no migration of the ³H cortisol into the skin from either formulation. The time was increased to 24 hours.

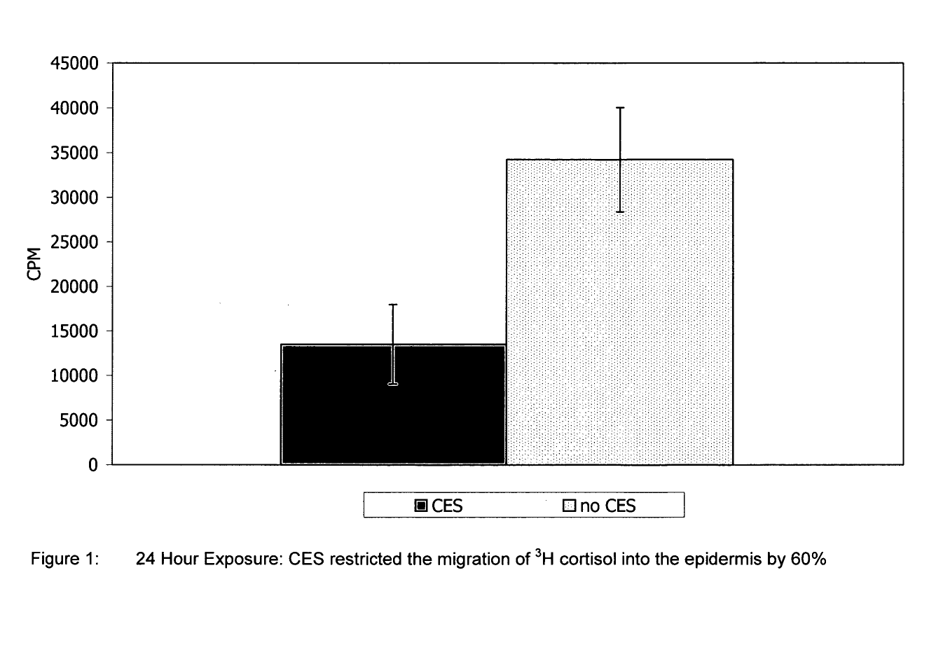

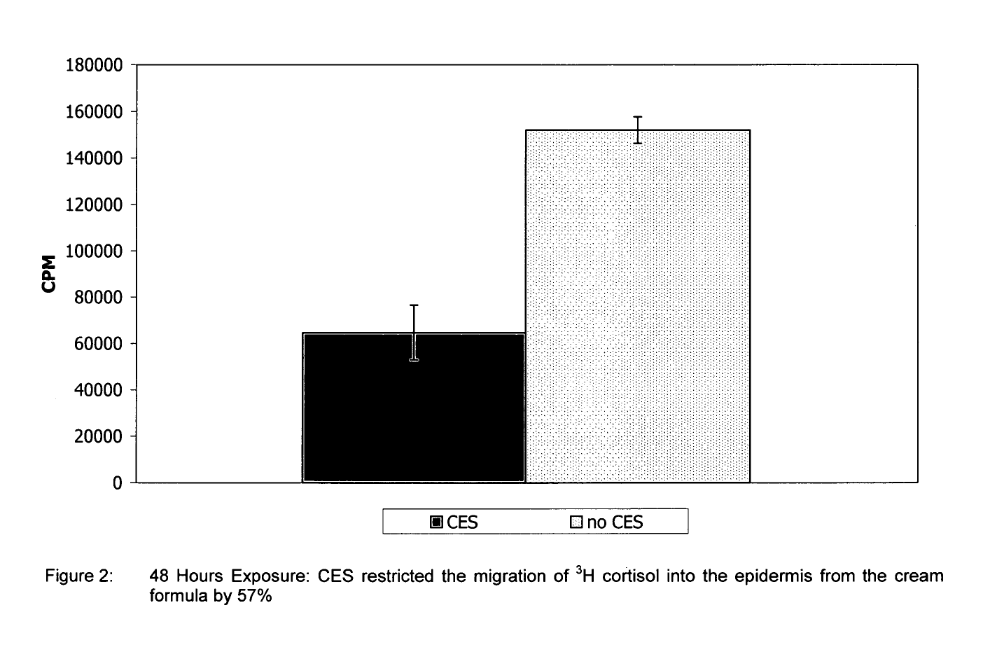

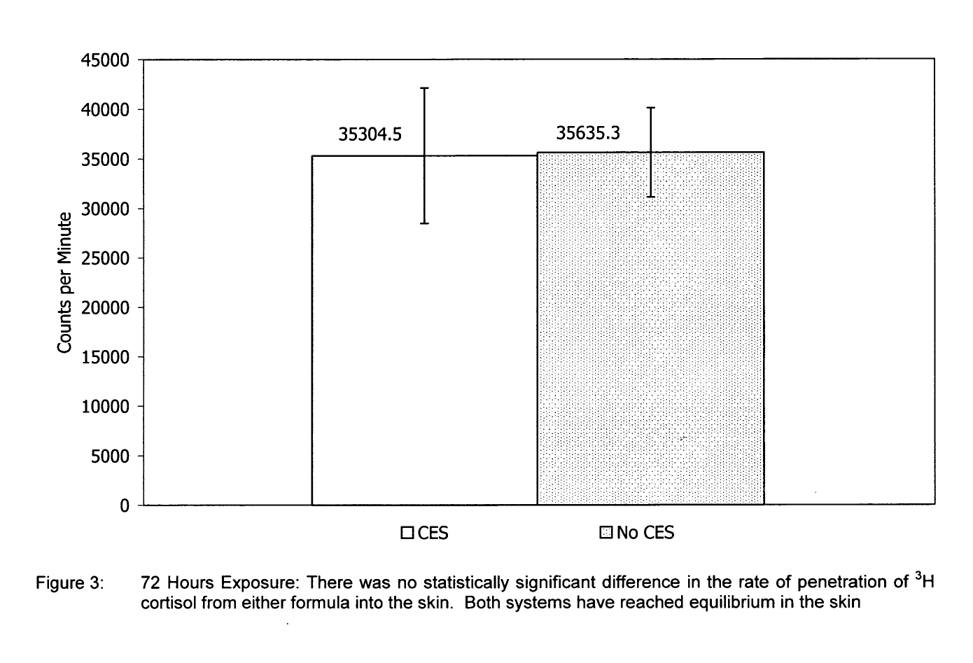

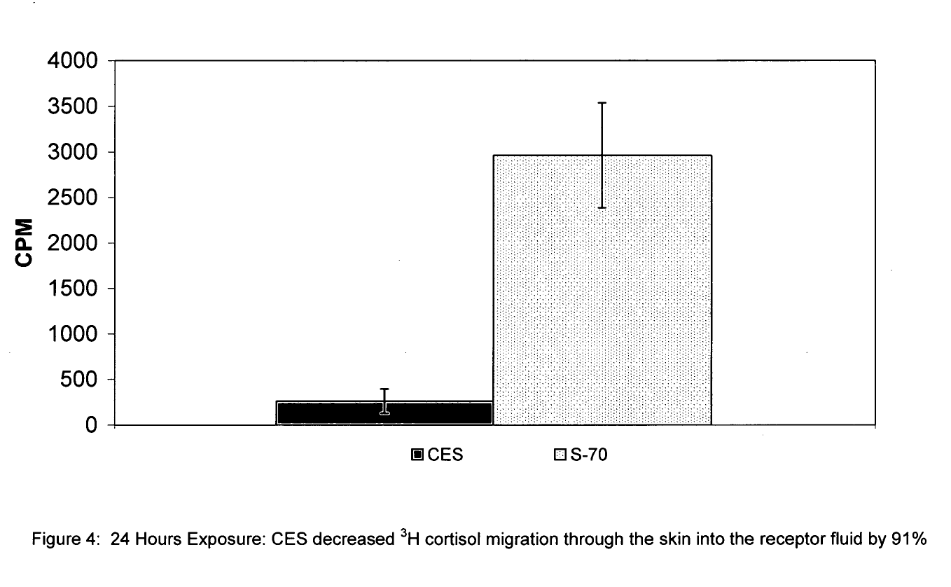

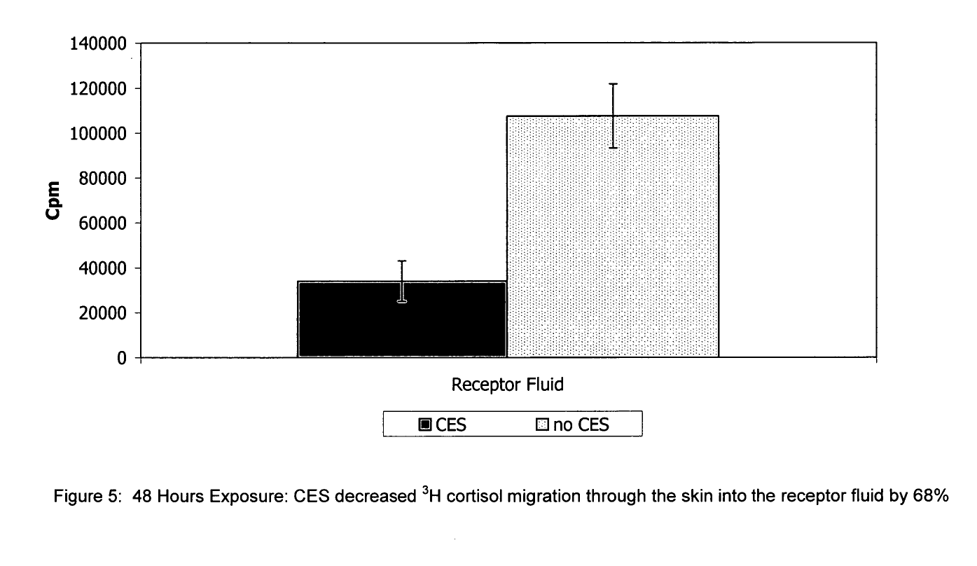

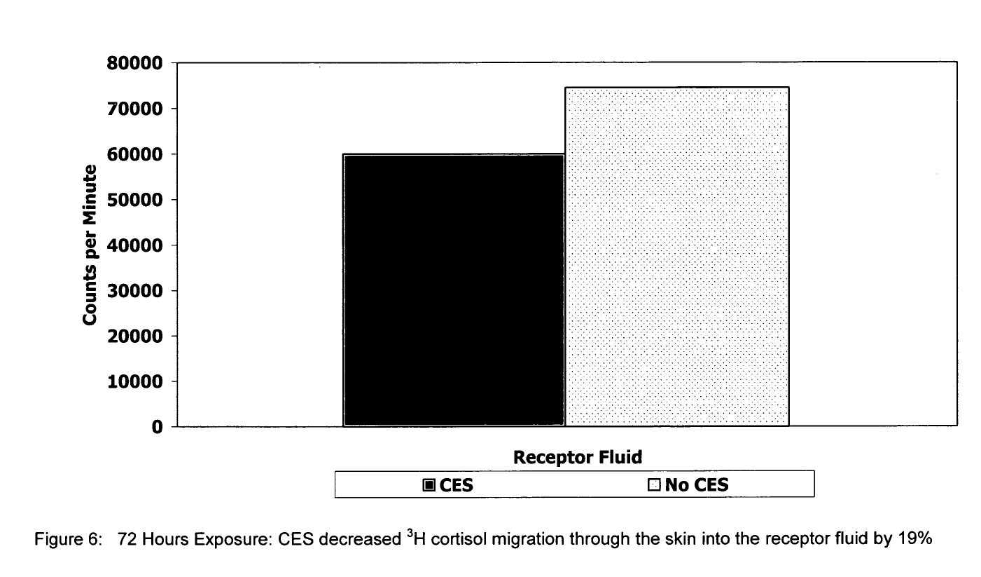

After 24 hours there was significantly less radiolabel found in both epidermis (FIG. 1) and the receptor fluid (FIG. 4) of the CES treated skin versus the matched S-70 control. It can be seen in FIG. 1 that the CES restricts the migration of the radiolabeled cortisol to the epidermis by 60% compared to the S-70 control. In FIG. 4, at 24 hours there was 91% less radiolabel in the CES treated skin receptor fluid as compared to the S-70 formula treated skin. During the next 24 hours (FIG. 2, 48H), the migration of the ³H cortisol into the CES treated epidermis was still significantly less than the S-70 treated skin. The CES restricted the migration by 57% as compared to the S-70 control (FIG. 2). When the receptor fluid data is viewed in FIG. 5, the amount of ³H cortisol found was significantly lower in the CES treated skin than the S-70 control by 68%. By 72H there was no significant difference in the amount of ³H cortisol found in the epidermis of either skin sample (FIG. 3). It appears that the two systems have achieved equilibrium in the split thickness skin. But, in FIG. 6 it appears that CES decreases the ³H cortisol release from the skin into the receptor fluid by 19% as compared to S-70. Note that this data is not cumulative. Each data set at each time period reflects the discreet differences between the CES containing material and the control at that time point.

From these data it can be concluded that the inclusion of CES into the formula increases the residence time of ³H cortisol in the skin. The increased residence time of ³H cortisol in the epidermis would correlate with less applications of the drug which would increase patient compliance, directed delivery of the drug and lower systemic exposure.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A topical pharmaceutical preparation exhibiting decreased flux or increased skin retention comprising: a topical active pharmaceutically ingredient in an amount of at least about 0.1% by weight of the final preparation, about 0.1 to about 20% by weight of mixed fatty alcohol phosphate esters comprising at least one alkoxylated fatty alcohol phosphate ester and at least one non-alkoxylated fatty alcohol phosphate ester present in a ration of 80:20 to 20:80 and a vehicle, said preparation having an improved flux or increased skin retention time of at least about 10% in 24 hours when compared to the same preparation without said mixed fatty alcohol phosphate esters.

2. The topical pharmaceutical preparation of claim 1 wherein said vehicle is an oil-in-water, water-in-oil or oil-in-oil emulsion.

3. The topical pharmaceutical preparation of claim 1 wherein said topical active pharmaceutically ingredients are vitamins, insect repellents, bioflavonoids, squalenes, salicylic acids, resouncinols, miconazoles, (N,N, diethyl-m-toluamide), tocopherols, tocopherol acetateS, retinoic acids, retinols, retinoids, zinc, benzoyl peroxide, perhydrocyclopentanophenanthrene derivatives including steroids, androgens, progestational hormones, corticosteroids, and estrogen based compounds.

4. The topical pharmaceutical preparation of claim 1 wherein said topical active pharmaceutically ingredient are hydrocortisone, salicylic acid, resouncinol, miconazole, DEET (N,N, diethyl-m-toluamide), tocopherol, tocopherol acetate, retinoic acid, retinol, hydrocortisone, retinoids, sulfur, erythromycin, zinc, and benzoyl peroxide.

5. The topical pharmaceutical preparation of claim 1 further comprising ³H cortisol.

6. The topical pharmaceutical preparation of claim 1 wherein said topical active pharmaceutically ingredient is present in an amount of between about 0.1 and about 50% by weight.

7. The topical pharmaceutical preparation of claim 1 further comprising at least one of an emollient, an emulsifier, a thickener, water, a preservative, a stabilizer, a pH adjusting substance, a color, a solvent, a cosolvent, a dispersion aid, a solid particulate.

8. The topical pharmaceutical preparation of claim 1 whose form is that of a cream, milk, lotion, gel, salve, ointment, spray, mousse, liquid, stick.

9. The topical pharmaceutical preparation of claim 1 further comprising: a gauze, pad, swab, cotton ball, batting, bandage, patch or occlusive barrier.

10. A pharmaceutical preparation exhibiting decreased flux or increased skin retention comprising: a active pharmaceutically ingredient in an amount of at least about 0.1% by weight of the final preparation, about 0.1 to about 20% by weight of mixed fatty alcohol phosphate esters comprising at least one alkoxylated fatty alcohol phosphate ester and at least one non-alkoxylated fatty alcohol phosphate ester present in a ration of 80:20 to 20:80 and a vehicle, said preparation having an improved flux or increased skin retention time of at least about 10% in 24 hours when compared to the same preparation without said mixed fatty alcohol phosphate esters.

11. The pharmaceutical preparation of claim 10 wherein said vehicle is an oil-in-water, water-in-oil or oil-in-oil emulsion.

12. The pharmaceutical preparation of claim 10 wherein said active pharmaceutically ingredients are vitamins, insect repellents, bioflavonoids, squalenes, salicylic acids, resouncinols, miconazoles, (N,N, diethyl-m-toluamide), tocopherols, tocopherol acetate, retinoic acids, retinols, retinoids, zinc, benzoyl peroxide, perhydrocyclopentanophenanthrene derivatives including steroids, androgens, progestational hormones, corticosteroids, and estrogen based compounds.

13. The pharmaceutical preparation of claim 10 wherein said active pharmaceutically ingredient are hydrocortisone, salicylic acid, resouncinol, miconazole, DEET (N,N, diethyl-m-toluamide), tocopherol, tocopherol acetates, retinoic acid, retinol, hydrocortisone, retinoids, sulfur, erythromycin, zinc, and benzoyl peroxide.

14. The pharmaceutical preparation of claim 10 further comprising ³H cortisol.

15. The pharmaceutical preparation of claim 10 wherein said active pharmaceutically ingredient is present in an amount of between about 0.1 and about 50% by weight.

16. The pharmaceutical preparation of claim 10 further comprising at least one of an emollient, an emulsifier, a thickener, water, a preservative, a stabilizer, a pH adjusting substance, a color, a solvent, a cosolvent, a dispersion aid, a solid particulate.

17. The pharmaceutical preparation of claim 10 whose form is that of a cream, milk, lotion, gel, salve, ointment, spray, mousse, liquid, stick.

18. The pharmaceutical preparation of claim 10 further comprising: a gauze, pad, swab, cotton ball, batting, bandage, patch or occlusive barrier.

19. A method of treating a topical condition in a patient in need thereof comprising: applying to an afflicted area of a patient the composition of claim 1, maintaining said composition in contact with said afflicted area of said patient, and optionally reapplying said formulation, for a time sufficient to treat said topical condition.
